(19) United States
(12) Patent Application Publication
(10) Pub. No.: US 2021/0236432 A1
(43) Pub. Date: Aug. 5, 2021

(54) COMPOSITIONS COMPRISING ROFLUMILAST FOR TREATING HIDRADENITIS SUPPURATIVA AND PRURIGO NODULARIS

(71) Applicant: Sol-Gel Technologies Ltd., Ness Ziona (IL)

(72) Inventors: Moshe Arkin, Kfar Shmaryahu (IL); Marcel Zighelboim, Kiryat Motzkin (IL); Karine Neimann, Ness Ziona (IL); Hila Hakak Djerbi, Tel Aviv (IL)

(73) Assignee: Sol-Gel Technologies Ltd., Ness Ziona (IL)

(21) Appl. No.: 17/166,336

(22) Filed: Feb. 3, 2021

Related U.S. Application Data
(60) Provisional application No. 63/069,850, filed on Aug. 25, 2020.
(60) Provisional application No. 62/969,206, filed on Feb. 3, 2020.

(51) Int. Cl. A61K 9/50; A61K 31/44; A61K 9/06

(52) U.S. Cl. CPC A61K 9/50; A61K 9/06; A61K 31/44

(57) ABSTRACT

Provided herein is a topical composition comprising roflumilast, roflumilast N-oxide or salts thereof as an active agent. The active agent in the composition of this invention is in encapsulated or non-encapsulated form, according to need.

The above composition is useful for the treatment, prevention or alleviation of a skin disorder selected from Hidradenitis suppurativa and Prurigo nodularis.

### CROSS-REFERENCE TO RELATED APPLICATIONS

This application claims the benefit of U.S. Provisional Application No. 63/069,850, filed Aug. 25, 2020 and of U.S. Provisional Application No. 62/969,206, filed Feb. 3, 3020 which are hereby incorporated in their entirety by reference.

### FIELD OF THE INVENTION

The present disclosure, in some embodiments, relates to a method of treatment of a skin disorder by topical administration to a subject in need thereof a composition comprising roflumilast as an active agent. The compositions of this invention are useful for the treatment, prevention or amelioration of skin disorders selected from Hidradenitis Suppurativa (HS), also known as acne inversa, and Prurigo Nodularis (PN), also known as nodular prurigo.

### BACKGROUND

Skin disorders (also known as skin conditions) in general and Hidradenitis Suppurativa (HS), and Prurigo Nodularis (PN) in particular vary greatly in symptoms and in severity. They are commonly treated with systemic and/or topical medicaments. Topical medicaments, while not always available, have the advantage of avoiding systemic side-effects. On the other hand, topical skin disorder treatments are sometimes accompanied by undesirable side-effects, especially at high doses.

Hidradenitis suppurativa (HS), also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” (Am. J. Clin. Dermatol., 2014, vol. 16. No. 1, pages 61-65). The clinical picture of HS includes solitary nodules, diffuse, painful abscesses, malodorous drainage, sinus tract formation and scarring. The exact cause of hidradenitis suppurativa is still unclear, but it is believed that the underlying mechanism involves dysfunction of the apocrine sweat glands or hair follicles.

There is no cure for HS, but treatments with drugs selected from oral antibiotics, corticosteroid injections, antiandrogen therapy with high dosages of cyproterone acetate and ethynyl estradiol, TNF inhibitors like adalimumab and immunosuppressive drugs have been attempted.

Prurigo nodularis (PN) is a skin disease characterized by pruritic (itchy) nodules which usually appear on the arms or legs. Patients often present with multiple excoriated lesions caused by scratching. Prurigo nodularis is very hard to treat, but current therapies include steroids, vitamins, cryosurgery, thalidomide and UVB light.

### SUMMARY OF THE INVENTION

This invention provides a topical composition comprising from about 0.01% w/w to about 1.0% w/w roflumilast, roflumilast N-oxide or salts thereof, and a carrier suitable for topical administration. The roflumilast, roflumilast N-oxide or salts thereof, in the composition of this invention is in encapsulated or non-encapsulated form, according to need.

In some embodiments, the composition of this invention is useful for the treatment, prevention or alleviation of a skin disorder selected from Hidradenitis suppurativa (HS) and Prurigo nodularis (PN).

### DETAILED DESCRIPTION OF THE INVENTION

The present invention provides topical compositions comprising roflumilast, roflumilast N-oxide or salts thereof, and methods of treatment useful for the treatment, prevention and amelioration of a skin disorder selected from HS and PN.

It occurred to the present inventors, that topical compositions comprising roflumilast, roflumilast N-oxide or salts thereof as a sole active agent may allow treatment of skin disorders for longer periods of time and exhibit improved therapeutic effects.

In some embodiments, there is provided a topical composition comprising from about 0.01% w/w to about 1.0% w/w roflumilast, roflumilast N-oxide or salts thereof, and a carrier suitable for topical administration.

In some embodiments, there is provided a topical composition comprising from about 0.01% w/w to about 0.1% w/w roflumilast, roflumilast N-oxide or salts thereof, and a carrier suitable for topical administration.

In some embodiments, there is provided a topical composition comprising from about 0.1% w/w to about 1.0% w/w roflumilast, roflumilast N-oxide of or salts thereof, and a carrier suitable for topical administration.

In some embodiments, the carrier suitable for topical administration does not comprise hexylene glycol or diethylene glycol monoethyl ether.

In some embodiments, there is provided a topical composition comprising from about 0.01% w/w to about 1.0% w/w roflumilast, roflumilast N-oxide or salts thereof, and a carrier suitable for topical administration, wherein the carrier suitable for topical administration does not comprise hexylene glycol or diethylene glycol monoethyl ether.

In some embodiments, there is provided a composition comprising roflumilast, roflumilast N-oxide or salts thereof, as an active agent, wherein the active agent is fully dissolved or partly dissolved and partly suspended in a carrier comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof (see, e.g., Examples 1-3 below).

In some embodiments, there is provided a composition comprising roflumilast, roflumilast N-oxide or salts thereof, as an active agent, wherein the active agent is encapsulated.

In some embodiments, there is provided a composition comprising roflumilast, roflumilast N-oxide or salts thereof, wherein the active agent of the composition is encapsulated within silicone dioxide (SiO₂) shell (see, e.g., Example 4 below).

The advantages of the active agent encapsulation are on the one hand the improved chemical stability of the active agent in the composition and on the other hand its improved therapeutic effect, including reduced side-effects and sustained-release effect.

### Active Agents in the Compositions of This Disclosure Roflumilast (a PDE4 Inhibitor)

Phosphodiesterase Type 4 inhibitors are blocking the degradation of cyclic adenosine monophosphate (cAMP) by phosphodiesterase 4 (PDE4). Some of the known PDE4 inhibitors are roflumilast, apremilast, crisaborole, rolipram, cilomilast, ibudilast and piclamilast.

Roflumilast was the first FDA-approved PDE4 inhibitor. This PDE4 inhibitor is FDA-approved (Daliresp®) and EMA-approved (Daxas®) for oral treatment of severe chronic obstructive pulmonary disease (COPD).

The systemic administration of roflumilast is accompanied by side-effects including inter aliaemesis and diarrhea, which is a big drawback to the oral administration. The topical administration of roflumilast comprising drugs provided in this disclosure avoids the systemic side-effects.

Roflumilast has a very low calculated water solubility (0.0062 mg/mL, according to the roflumilast DrugBank monograph), which is a problem for a topical product.

The following routes for improving the roflumilast solubility and skin permeability are put forward in this disclosure:
a. Use of potent solvents, like DMSO, propylene glycol, a polyethylene glycol (PEG), ethanol, isopropyl alcohol, dimethyl isosorbide, isopropyl myristate, oleic acid, glycofurol and combinations thereof (see, e.g., Examples 1-3 below).
b. Use of finely milled or micronized roflumilast.
c. Use a soluble roflumilast salt as active agent.
d. Use of a soluble roflumilast pro-drug.
e. Use of a roflumilast-cyclodextrin complex.
f. Use of an amorphous form of roflumilast.
g. Use of a roflumilast solid dispersion.

The term “Roflumilast” as used herein includes roflumilast free base, its pharmacologically active metabolites including roflumilast N-oxide or salt thereof, or roflumilast pharmaceutically acceptable salt thereof. Preferably, roflumilast is roflumilast free base. Roflumilast present in the composition according to present invention can be in crystalline form or amorphous form.

Suitable salts for roflumilast or salts of roflumilast N-oxide include inorganic and organic acids. In other embodiments, non-limited examples of acids include: hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation of roflumilast.

According to some embodiments of the present invention, the coated/encapsulated form of the active agent (roflumilast, roflumilast N-oxide or salts thereof) may be in form of a polymeric microsponge/silica microsphere where the active agent is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety.

In other embodiments, the coated/encapsulated form of the active agent (roflumilast, roflumilast N-oxide or salts thereof) may be in form of microcapsules which are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 20060003014, US 2010/180464; which are incorporated herein by reference in their entirety.

### Methods of Treatment

According to an aspect of the invention, there is provided a method of treatment of a skin disorder selected from HS and PN, by treatment of a subject in need thereof with a composition of roflumilast, roflumilast N-oxide or pharmaceutically acceptable salt thereof as an active agent.

In some embodiments, the effective amount is a therapeutically effective amount of roflumilast, roflumilast N-oxide or salts thereof, namely an amount which will cure, treat, alleviate or prevent a skin disorder selected from HS and PN.

According to an aspect of the invention, there is provided a method of treating hidradenitis suppurativa (HS) in a subject, the method comprises administering a topical composition comprising roflumilast, roflumilast N-oxide or salts thereof. In another embodiment, the concentration of the roflumilast, roflumilast N-oxide or salts thereof is between about 0.01% to 1% w/w. In another embodiment, the roflumilast, roflumilast N-oxide or salts thereof is the sole active agent.

According to an aspect of the invention, there is provided a method of treating prurigo nodularis (PN) in a subject, the method comprises administering a topical composition comprising roflumilast, roflumilast N-oxide or salts thereof. In another embodiment, the concentration of the roflumilast, roflumilast N-oxide or salts thereof is between about 0.01% to 1% w/w. In another embodiment, the roflumilast, roflumilast N-oxide or pharmaceutically acceptable salt thereof is the sole active agent.

### Regimen of Administration of the Topical Compositions Comprising Roflumilast

Therapeutically effective concentrations of the active agents in the compositions of this invention for treatment, prevention or alleviation of the symptoms manifested by a skin disorder are determined by empirical methods known in the art.

Exemplary dosages, strengths and concentrations of roflumilast, roflumilast N-oxide or salts thereof in the topical compositions of this invention, are in the range of from about or at 0.01%, 0.05%, 0.1%, 0.25%, 0.3%, 0.5%, 0.75% or 1% w/w. Typical strengths in the topical compositions of this invention are 0.01%, 0.1% or 1% w/w roflumilast.

The frequency of administration can be determined empirically. Exemplary frequencies are once daily, twice daily, weekly, bi-weekly or monthly.

Typical administration frequencies of the topical compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### Kits

Kits containing the compositions optionally including instructions for administration are provided. The kits include, for example, the compositions as provided herein. Additionally, provided herein are kits containing the above-described compositions and optionally instructions for administration by topical, transdermal, or other routes, depending on the composition to be delivered.

The compositions provided herein can be packaged as articles of manufacture containing packaging material, a composition provided herein, and a label that indicates that the composition is for treating a skin disorder and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Embodiments

In some embodiments, there is provided a topical composition comprising from about 0.01% w/w to about 1.0% w/w roflumilast, roflumilast N-oxide or salts thereof, and a carrier suitable for topical administration.

In some embodiments, there is provided a topical composition comprising from about 0.01% w/w to about 0.1% w/w roflumilast, roflumilast N-oxide or salts thereof, and a carrier suitable for topical administration, wherein the carrier does not include hexylene glycol or diethylene glycol monoethyl ether.

In some embodiments, there is provided a topical composition comprising from about 0.1% w/w to about 1.0% w/w roflumilast, roflumilast N-oxide or salts thereof, and a carrier suitable for topical administration, wherein the carrier does not include hexylene glycol or diethylene glycol monoethyl ether.

In some embodiments, there is provided a topical composition comprising from about 0.01% w/w to about 1% w/w roflumilast, roflumilast N-oxide or salts thereof, and a carrier suitable for topical administration, wherein the carrier does not include hexylene glycol or diethylene glycol monoethyl ether.

In some embodiments, there is provided a topical composition comprising from about 0.01% w/w to about 1.0% w/w roflumilast, roflumilast N-oxide or salts thereof, and a carrier suitable for topical administration, wherein the roflumilast, roflumilast N-oxide or salts thereof is encapsulated.

In some embodiments, there is provided a topical composition comprising from about 0.01% w/w to about 1.0% w/w of encapsulated or non-encapsulated roflumilast, roflumilast N-oxide or salts thereof, and a carrier suitable for topical administration, wherein the roflumilast fully dissolved or partly dissolved or partly suspended in the carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

In one embodiment, this invention provides a topical composition comprising from about 0.01% w/w to about 1.0% w/w roflumilast, roflumilast N-oxide or salts thereof, and a carrier suitable for topical administration. In another embodiment the roflumilast, roflumilast N-oxide or salts thereof is encapsulated. In another embodiment the roflumilast, roflumilast N-oxide or salts thereof is not encapsulated.

In one embodiment, this invention provides a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether. In another embodiment the roflumilast, roflumilast N-oxide or salts thereof is encapsulated. In another embodiment the roflumilast, roflumilast N-oxide or salts thereof is not encapsulated.

In one embodiment, this invention is directed to a topical composition comprising from about 0.01% w/w to about 1.0% w/w roflumilast, roflumilast N-oxide or salts thereof, and a carrier suitable for topical administration; wherein the roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof. In another embodiment the roflumilast, roflumilast N-oxide or salts thereof is encapsulated. In another embodiment the roflumilast, roflumilast N-oxide or salts thereof is not encapsulated.

In one embodiment, this invention is directed to a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether; wherein the roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof. In another embodiment the roflumilast, roflumilast N-oxide or salts thereof is encapsulated. In another embodiment the roflumilast, roflumilast N-oxide or salts thereof is not encapsulated.

In some embodiments, there is provided a dosage form comprising a composition of the present invention, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

In one embodiment, this invention provides a dosage form comprising a topical composition comprising from about 0.01% w/w to about 1.0% w/w roflumilast, roflumilast N-oxide or salts thereof, and a carrier suitable for topical administration, wherein the composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe. In another embodiment the roflumilast, roflumilast N-oxide or salts thereof is encapsulated. In another embodiment the roflumilast, roflumilast N-oxide or salts thereof is not encapsulated. In another embodiment, roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

In one embodiment, this invention provides a dosage form comprising a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether, wherein the composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe. In another embodiment the roflumilast, roflumilast N-oxide or salts thereof is encapsulated. In another embodiment the roflumilast, salts of roflumilast, the N-oxide of roflumilast or salts thereof is not encapsulated. In another embodiment, roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

In some embodiments, there is provided a method of treatment, prevention or alleviation of a skin disorder selected from HS and PN, the method comprises topical administration to the affected area of a subject with the skin disorder a therapeutically effective amount of a composition of the present invention. In some embodiments, there is provided a method of treatment, prevention or alleviation of a skin disorder selected from HS and PN, the method comprises topical administration to the affected area of a subject with the skin disorder a therapeutically effective amount of a composition comprising from about 0.01% w/w to about 1.0% w/w roflumilast, roflumilast N-oxide or salts thereof, and a carrier suitable for topical administration.

In some embodiments, there is provided a method of treatment, prevention or alleviation of a skin disorder selected from HS and PN, wherein the treatment comprises once daily or twice daily topical administration to a subject in need thereof of a therapeutically effective amount of a composition of the present invention. In some embodiments, the composition comprises from about 0.01% w/w to about 1.0% w/w roflumilast, roflumilast N-oxide or salts thereof, and a carrier suitable for topical administration.

In one embodiment, this invention provides a method of treatment, prevention or alleviation of a skin disorder selected from Hidradenitis suppurativa (HS) and Prurigo nodularis (PN), said method comprises topical administration to the affected area of a subject with the skin disorder a therapeutically effective amount of a topical composition comprising from about 0.01% w/w to about 1.0% w/w roflumilast, roflumilast N-oxide or salts thereof, and a carrier suitable for topical administration. In another embodiment the roflumilast, roflumilast N-oxide or salts thereof is encapsulated. In another embodiment the roflumilast, roflumilast N-oxide or salts thereof is not encapsulated. In another embodiment, roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

In one embodiment, this invention provides a method of treatment, prevention or alleviation of a skin disorder selected from Hidradenitis suppurativa (HS) and Prurigo nodularis (PN), said method comprises topical administration to the affected area of a subject with the skin disorder a therapeutically effective amount of a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether. In another embodiment the roflumilast, roflumilast N-oxide or salts thereof is encapsulated. In another embodiment the roflumilast, roflumilast N-oxide or salts thereof is not encapsulated. In another embodiment, roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

In one embodiment, this invention provides a method of treatment, prevention or alleviation of a skin disorder selected from Hidradenitis suppurativa (HS) and Prurigo nodularis (PN), said method comprises topical administration to the affected area of a subject with the skin disorder a therapeutically effective amount of a composition of this invention, wherein the treatment comprises once daily or twice daily topical administration to a subject in need thereof of a therapeutically effective amount of a composition of this invention.

In one embodiment, this invention provides a method of treatment, prevention or alleviation of Hidradenitis suppurativa (HS), said method comprises topical administration to the affected area of a subject with the Hidradenitis suppurativa (HS) a therapeutically effective amount of a composition of this invention, wherein the treatment comprises once daily or twice daily topical administration to a subject in need thereof of a therapeutically effective amount of a composition of this invention. In another embodiment, the composition is a topical composition comprising from about 0.01% w/w to about 1.0% w/w roflumilast, roflumilast N-oxide or salts thereof, and a carrier suitable for topical administration. In another embodiment, the composition is a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether. In another embodiment the roflumilast, roflumilast N-oxide or salts thereof is encapsulated. In another embodiment the roflumilast, roflumilast N-oxide or salts thereof is not encapsulated.

In one embodiment, this invention provides a method of treatment, prevention or alleviation of Prurigo nodularis (PN), said method comprises topical administration to the affected area of a subject with the Prurigo nodularis (PN) a therapeutically effective amount of a composition of this invention, wherein the treatment comprises once daily or twice daily topical administration to a subject in need thereof of a therapeutically effective amount of a composition of this invention. In another embodiment, the composition is a topical composition comprising from about 0.01% w/w to about 1.0% w/w roflumilast, roflumilast N-oxide or salts thereof, and a carrier suitable for topical administration. In another embodiment, the composition is a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether. In another embodiment the roflumilast, roflumilast N-oxide or salts thereof is encapsulated. In another embodiment the roflumilast, roflumilast N-oxide or salts thereof is not encapsulated.

In some embodiments, there is provided a regimen of administration comprising the once daily or twice daily administration to an affected area of a subject with a skin disorder a therapeutically effective dose of the composition of the present invention until the skin disorder is cured, prevented or alleviated. In some embodiments, the composition is a topical composition comprising from about 0.01% w/w to about 1.0% w/w roflumilast, roflumilast N-oxide or salts thereof, and a carrier suitable for topical administration. In another embodiment, the composition is a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether. In another embodiment the roflumilast, roflumilast N-oxide or salts thereof is encapsulated. In another embodiment the roflumilast, roflumilast N-oxide or salts thereof is not encapsulated. In another embodiment, the skin disorder is Hidradenitis suppurativa (HS) or Prurigo nodularis (PN).

In some embodiments, there is provided a regimen of administration comprising the once daily or twice daily administration to a subject with a skin disorder a therapeutically effective amount of a dosage form comprising a composition of the present invention until the skin disorder is cured, prevented or alleviated, wherein the composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe. In another embodiment, the composition is a topical composition comprising from about 0.01% w/w to about 1.0% w/w roflumilast, roflumilast N-oxide or salts thereof, and a carrier suitable for topical administration. In another embodiment, the composition is a topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether. In another embodiment the roflumilast, roflumilast N-oxide or salts thereof is encapsulated. In another embodiment the roflumilast, roflumilast N-oxide or salts thereof is not encapsulated. In another embodiment, the skin disorder is Hidradenitis suppurativa (HS) or Prurigo nodularis (PN).

In some embodiments, there is provided a kit comprising one or more dosage forms comprising the composition of the present invention and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in in a single embodiment. Conversely, various features of the invention, which are, for brevity, described in the context of a single embodiment, may also be provided separately or in any suitable sub- or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

In the examples below, all % values referring to a solution are in (w/w).

All % values, referring to dispersions (suspensions) are in (w/w).

Unless otherwise indicated, all solutions used in the example below refer to an aqueous solution of the indicated ingredient.

All the exemplary s of specific active agents comprise finely milled or micronized roflumilast, at least one additional active agent selected from a retinoid (selected from tretinoin, trifarotene, adapalene and tazarotene), benzoyl peroxide (BPO) and s thereof.

Roflumilast in the exemplary compositions may be used as such, as detailed in Examples 1-3, or encapsulated, as detailed in Example 4.

All active agents in the compositions of Example 4 are SiO₂-encapsulated.

### EXAMPLES

### Example 1

### General Procedure For the Preparation of a Composition Comprising 0.01% w/w Non-Encapsulated Roflumilast

TABLE 1
Composition comprising 0.01% w/w non-encapsulated Roflumilast

| Ingredient | % in formulation |
|---|---|
| Roflumilast | 0.01 |
| DMSO | 70 |
| Propylene glycol | 26.24 |
| 2-phenoxyethanol | 0.5 |
| Methylparaben | 0.25 |
| Carbopol 980 | 3.0 |

Procedure: Roflumilast is dissolved in DMSO at 40° C.; Methylparaben is added under stirring; Carbopol is added under stirring; 2-phenoxyethanol is dissolved in propylene glycol and added; The formulation is stirred and homogenized to obtain a homogeneous gel; The active agent in the composition is fully dissolved or partly dissolved or partly suspended.

### Example 2

### General Procedure For the Preparation of a Composition Comprising 0.1% w/w Roflumilast

TABLE 2
Composition comprising 0.1% w/w non-encapsulated Roflumilast

| Ingredient | % in formulation |
|---|---|
| Roflumilast | 0.1 |
| DMSO | 70 |
| Propylene glycol | 26.15 |
| 2-phenoxyethanol | 0.5 |
| Methylparaben | 0.25 |
| Carbopol 980 | 3.0 |

Procedure: Roflumilast is dissolved in DMSO at 40° C.; Methylparaben is added under stirring; Carbopol is added under stirring; 2-phenoxyethanol is dissolved in propylene glycol and added; The formulation is stirred and homogenized to obtain a homogeneous gel; The active agent in the composition is fully dissolved or partly dissolved or partly suspended.

### Example 3

General Procedure For the Preparation of a Composition Comprising 1% w/w Roflumilast

TABLE 3
Composition comprising 1% w/w non-encapsulated Roflumilast

| Ingredient | % in formulation |
|---|---|
| Roflumilast | 1 |
| DMSO | 70 |
| Propylene glycol | 25.25 |
| 2-phenoxyethanol | 0.5 |
| Methylparaben | 0.25 |
| Carbopol 980 | 3.0 |

Procedure: Roflumilast is dissolved in DMSO at 40° C.; Methylparaben is added under stirring; Carbopol is added under stirring; 2-phenoxyethanol is dissolved in propylene glycol and added; The formulation is stirred and homogenized to obtain a homogeneous gel; The active agent in the composition is fully dissolved or partly dissolved or partly suspended.

### Example 4

### General Procedure For the Preparation of a Cream Composition Comprising 0.01-1% w/w SiO₂-Encapsulated Roflumilast (E-Roflumilast)

Stage 1. Preparation of Encapsulated Roflumilast, 15% (E-Roflumilast Water Suspension)

a) Preparation of Roflumilast Suspension and Acid Cocktail

Roflumilast suspension is prepared by mixing 125.67 grams of CTAC CT-429 (Cetrimonium Chloride 30%), 2250 grams of Roflumilast, and 5200 grams water under high shear. The solution is homogenized for 60 minutes at 33° C. (no more than 45° C.), and then the pH of the solution is adjusted to 7.0 using sodium hydroxide solution (20%).

An acid cocktail is prepared using 493 grams Hydrochloric acid (37%), 98 grams anhydrous Citric Acid, 147 grams Lactic Acid (90%), and 794 grams water.

b) Coating Cycle

The coating cycle is started by adding 38 grams sodium silicate solution extra pure (28%) to the Roflumilast suspension prepared in step a) under high shear, followed by adding the acid cocktail prepared in step (a) to adjust the pH to be lower than 6.8, and followed by adding 57 grams PDAC (3%) solution to the mixture. The cycle is repeated 50 times while the mixture is stirred under high shear for 17 hours. After the 50 cycles, the pH of the mixture is adjusted to 5.0 using the acid cocktail, and water is added to complete the total weight of the mixture to 15 kilograms. The composition of the final Roflumilast water suspension product is shown in Table 4.

TABLE 4
Composition of the encapsulated Roflumilast 15% water suspension

| Ingredient | % of ingredient in the suspension |
|---|---|
| Polyquarternium-7 | 0.53 |
| Hydrochloric Acid | 0.87 |
| Citric Acid, Anhydrous | 0.46 |
| Lactic Acid | 0.63 |
| Silicon Dioxide | 3.42 |
| Sodium hydroxide | 0.01 |
| Cetrimonium Chloride | 0.25 |
| Roflumilast | 15.00 |
| Sterile Water for Irrigation | 78.83 |

Stage 2. Preparation of Cream Formulation of Encapsulated Roflumilast

Oil Phase: 720.0 grams of Cyclomethicone 5-N, 540.0 of grams Cetyl Alcohol, 360.0 grams Polyoxyl 100 Stearate, and 540.0 grams of Glyceryl Monostearate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added, and the resulting mixture is homogenized at 3300 rpm for 10 minutes to ensure that all materials completely melted and dissolved. 76.5 grams of sodium hydroxide (20%) are then added and the mixture is stirred under high shear for 10 minutes at no less than 70° C.

The oil phase is added to the water phase under high shear at 78° C., and the resulting emulsion is homogenized at 3300 rpm for 10 minutes.

300-3580 grams of encapsulated Roflumilast 15% water suspension made as described above is added to the emulsion at 65° C. and mixed at 1400 rpm for 10 minutes. The emulsion is cooled to 35° C., and the emulsion is stirred at 1400 rpm for 10 minutes. Water is added until the total weight of the cream reached 18 kilograms. The composition of the formulation prepared in this example is shown in Table 5.

TABLE 5
Composition of Cream Formulation of Encapsulated Roflumilast

| | % of pure ingredient in the |
|---|---|
| Ingredient | composition |
| Polyquarternium-7 | 0.21 |
| Hydrochloric Acid | 0.51 |
| Citric Acid, Anhydrous | 0.18 |
| Lactic Acid | 0.25 |
| Silicon Dioxide | 1.44 |
| Sodium hydroxide | 0.09 |
| Cetrimonium Chloride | 0.11 |
| Roflumilast | 0.01-1.0 |
| Beeswax | 0.04 |
| Squalane | 0.28 |
| Ethanol (Alcohol) | 0.14 |
| Butylated hydroxytoluene | 0.02 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | Up to 100% |

### Example 5

Preparation of a Cream Composition Comprising 0.3% Roflumilast

TABLE 6
Composition of roflumilast 0.3%

| # | Excipient | w/w % |
|---|---|---|
| 1. | Purified water | 22.45 |
| 2. | Buffer citric acid (pH = 5.5) | 22.00 |
| 3. | MYRJ S100 | 1.80 |
| 4. | Methylparaben | 0.20 |
| 5. | Propylparaben | 0.05 |
| 6. | Petrolatum white | 10.00 |
| 7. | Isopropyl myristate | 5.00 |
| 8. | Cetostearyl alcohol | 3.00 |
| 9. | Cetyl alcohol | 3.00 |
| 10. | Imwitor 900 k | 2.20 |
| 11. | PEG 400 | 30.00 |
| 12. | Roflumilast | 0.30 |

Water phase: Water, citric acid buffer, MYRJ S100, methylparaben and propylparaben were weighted into a glass beaker. The beaker was placed inside a hot water bath adjusted to 70-75° C., and the mixture was mixed with a magnetic stirrer until a clear solution was obtained

Oil phase In a separate glass beaker—petrolatum, isopropyl myristate, cetostearyl alcohol, cetyl alcohol and imwitor 900k were weighted. The beaker was placed inside a hot water bath adjusted to 70-75° C., and the mixture was mixed with a magnetic stirrer until clear solution was obtained.

Active phase: In a separate glass beaker PEG 400 was weighed Then, roflumilast was added gradually and the solution was mixed with a magnetic stirrer until a clear solution free from particles was obtained. If the roflumilast didn't dissolve, the beaker was placed inside a hot water bath adjusted to 60° C. for a few minutes until a clear solution was obtained.

The oil phase was added to the water phase while mixing and homogenizing for about 5 minutes. The emulsion was cooled down.

Once the temperature reached 45-50° C., the roflumilast solution was added to the emulsion while mixing and homogenizing for about 5 minutes. Then water was added for batch completion and the mixture was continued to cool down until a homogenous cream was obtained.

What is claimed is:

1. A topical composition comprising from about 0.01% w/w to about 1.0% w/w roflumilast, roflumilast N-oxide or salts thereof, and a carrier suitable for topical administration.

2. A topical composition comprising from about 0.01% w/w to about 1.0% w/w roflumilast, roflumilast N-oxide or salts thereof, and a carrier suitable for topical administration, wherein the carrier does not comprise hexylene glycol or diethylene glycol monoethyl ether.

3. The composition of claim 1, wherein the roflumilast, roflumilast N-oxide or salts thereof, is encapsulated.

4. The composition of claim 2, wherein the roflumilast, roflumilast N-oxide or salts thereof, is encapsulated.

5. The composition of claim 1, wherein the roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

6. The composition of claim 2, wherein the roflumilast, roflumilast N-oxide or salts thereof is fully dissolved or partly dissolved or partly suspended in a carrier suitable for topical administration comprising at least one potent solvent, selected from dimethylsulfoxide (DMSO), propylene glycol, a polyethylene glycol (PEG), ethanol, isopropyl alcohol, dimethyl isosorbide, isopropyl myristate, oleic acid, glycofurol and combinations thereof.

7. A dosage form comprising the composition of claim 1, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

8. A dosage form comprising the composition of claim 2, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

9. A method of treatment, prevention or alleviation of a skin disorder selected from Hidradenitis suppurativa (HS) and Prurigo nodularis (PN), said method comprises topical administration to the affected area of a subject with the skin disorder a therapeutically effective amount of a composition of claim 1.

10. The method of claim 9, wherein the skin disorder is Hidradenitis suppurativa.

11. The method of claim 9, wherein the skin disorder is Prurigo nodularis.

12. The method of claim 9, wherein the treatment comprises once daily or twice daily topical administration to a subject in need thereof of a therapeutically effective amount of a composition comprising from about 0.01% w/w to about 1.0% w/w roflumilast, roflumilast N-oxide or salts thereof, and a carrier suitable for topical administration.

13. A regimen of administration comprising the once daily or twice daily administration to an affected area of a subject with a Hidradenitis suppurativa (HS) or Prurigo nodularis (PN) a therapeutically effective dose of the composition of claim 1 until the skin disorder is cured, prevented or alleviated.

14. A regimen of administration comprising the once daily or twice daily administration to a subject with a Hidradenitis suppurativa (HS) or Prurigo nodularis (PN) a therapeutically effective amount of a dosage form of claim 7.

15. A kit comprising one or more dosage forms of claim 7 and instructions for use.
